(19) United States
(12) Patent Application Publication
(10) Pub. No.: US 2005/0288716 A1
(43) Pub. Date: Dec. 29, 2005

(54) Piezoelectric devices and methods for restoring normal contraction rhythm of a heart

(76) Inventor: Otto Zsigmond, Kamloops (CA)

Correspondence Address: Otto Zsigmond, 702 - 198 Aquarius Mews, Vancouver, BC, V6Z 2Y4, CA

(21) Appl. No.: 10/862,321

(22) Filed: Jun. 8, 2004

(51) Int. Cl. A61N 1/39

(52) U.S. Cl. 607/5

(57) ABSTRACT

There is provided piezoelectric devices for restoring normal contraction rhythms of a heart, or for pacing a heart, comprising a housing, a piezoelectric generator seated within the housing and having first and second electrical conductors wherein the first conductor is connected to an electrically conducting portion of the housing, an electrically conductive skin contact electrode wherein the electrode is electrically insulated from the conducting portion of the housing and is connection with the second conductor, and an actuator for actuating the piezoelectric generator to generate an electrical current in response to stimuli from a user. There is further provided methods for restoring normal contraction rhythms of a heart in cardiac arrest, or for pacing a heart, comprising the administering an electric shock from a piezoelectric device to the chest area of a mammal proximal the heart. The use of a piezoelectric device for such functions is also provided.

### FIELD OF INVENTION

The present invention relates to devices and methods of reestablishing normal contraction rhythms in a heart that is not beating properly, and in particular, piezoelectric devices and methods of reestablishing normal contraction rhythms using electrical shock from a piezoelectric generating device.

### BACKGROUND OF THE INVENTION

It is recognized that the probability of surviving a heart attack often depends critically on the speed with which appropriate medical care is provided. One of the most common and life threatening consequences of a heart attack is the development of a cardiac arrhythmia such as ventricular fibrillation in which the heart cannot beat in a coordinated fashion and is unable to pump a significant volume of blood. When such an arrhythmia occurs, serious brain damage and death will invariably result unless a normal heart rhythm can be restored within a few minutes. The worst scenario is if the heart stops completely, known as asystole, which presents a much lower chance of resuscitation if it is not restarted immediately. A compounding factor is that most often, heart attacks occur in the home or in a family setting where immediate medical attention is not available.

The most effective treatment for ventricular fibrillation is the application of an electric shock to the victim. In ventricular fibrillation, electrical energy is present in the heart, but it is chaotic. If the heart can be shocked quickly with a defibrillator, a normal heart rhythm may be restored. By a mechanism not fully understood, such an electric shock frequently terminates the chaotic activity characteristic of arrhythmia, and restores the normal pumping action of the heart. If this shock is delivered within minutes after collapse, many victims can survive. The key is to be quick. Studies conducted at cardiac rehabilitation centers have shown that when sudden cardiac arrest victims in ventricular fibrillation or asystole receive defibrillation therapy within the first minute or two after collapse, a large majority survive to be discharged from the hospital. However, in more typical community settings, victims of sudden cardiac art rarely survive. This is because most victims do not have immediate access to prompt, definitive treatment. Too much time elapses before the defibrillator arrives—if it arrives at all.

Defibrillators for producing and delivering such shocks have been known and successfully used for many years. However, the size and cost of prior defibrillators coupled with the risk they pose if used improperly, have generally restricted the use of defibrillators to hospitals and to emergency medical facilities. Many lives would be saved each year if defibrillators could be made more immediately available to heart attack victims, high risk patients, heart patients and caregivers for same, and to the aging population in general

A large number of heart attacks occur to people who have a history of cardiac problems, and who are therefore known to be at risk. In recent years, many family members of high risk patients have received training in cardiopulmonary resuscitation, a technique designed to maintain some blood flow even if the heart is in fibrillation or has stopped beating altogether. Such training is helpful because a large percentage of repeat heart attacks occur in the presence of a family member. Unfortunately, it has to date not been possible to provide the family members of high risk patients with access to the generally more effective technique of defibrillation, because of the difficulty of designing a defibrillator that is portable and that can be safely and effectively used by non medical personnel. Accordingly, there is a need for such a device.

### SUMMARY OF THE INVENTION

I have discovered that the application of an electrical shock from a piezoelectric device, such as for example, a piezoelectric acupuncture device, to the upper portion of the left anterior chest of a patient whose heart is in cardiac arrest (i.e. ventricular fibrillation or asystole) has the effect of terminating the chaotic activity of the heart and restoring the normal pumping action of the heart. Alternatively, in addition to being able to restart a heart in cardiac arrest, electrical shocks from a piezoelectric device may also be used to manually pace a heart if the pumping action of the heart cannot be resort. Accordingly, the within invention is directed at piezoelectric devices which are capable of generating an electric shock for restoring normal contraction rhythms or defibrillating a heart, methods of using such piezoelectric devices for reestablishing normal contraction rhythms or defibrillating a heart, and the use of piezoelectric devices for such functions. The within invention is further directed at piezoelectric devices which are capable of generating an electric shock for pacing a heart, methods of using such piezoelectric devices for pacing a heart, and methods for pacing a heart using such piezoelectric devices. The method of the invention may be performed using the devices as described herein; however, any suitable piezoelectric device having the properties discussed herein may be used.

Accordingly, in some embodiments of the present invention, there is provided methods for the use of a piezoelectric shock generating device to restore normal contraction rhythms of a heart in cardiac arrest. In some embodiments, the piezoelectric device may be a piezoelectric acupuncture device such as those which are widely available in the marketplace. Some examples of such piezoelectric acupuncture devices are described in German Patent reference DE-A1-40 26 820 and published international application WO 01/01920, and each of these references is incorporated herein by reference. However, any piezoelectric device capable of delivering a suitable electrical shock may be used.

In some embodiments, the present invention also provides a piezoelectric defibrillator that is portable, easy to use, and comparatively inexpensive to manufacture. The piezoelectric defibrillator is sufficiently compact and lightweight to be kept at all times in the immediate vicinity of a person known to be at risk to heart attacks.

In some embodiments of the invention, the piezoelectric defibrillator comprises a piezoelectric generator inside an elongate, electrically conductive casing or housing. The piezoelectric generator is electrically insulated from the casing, and it has two electrical conductors or electrical wires: a positive and negative. One conductor is electrically connected to a contact pin which has a terminal treatment head or skin contact portion for contact with the skin. The contact pin and treatment head are also electrically insulated from the casing. The other conductor is electrically connected to the casing. The piezoelectric generator may be a push-button actuated, percussion type piezoelectric generator of the kind known in the art (these are also known as igniters in the prior art since they are often used to ignite flammable gases), which typically comprise a piezoelectric element and a spring loaded percussion mass connected to a pushbutton actuator. The percussion mass stores kinetic energy (usually as a spring is loaded) as the push button is depressed until a point is reached where the stored kinetic energy is released to suddenly and forcibly drive the percussion mass to strike the piezoelectric element, thereby generating an electric current as a result of physical compression of the piezoelectric element. In some embodiments, the piezoelectric generator may comprise a percussion mass capable of elastic displacement and provided with two diametrically opposite stubs displaceable on an incline made for that purpose on a push-button body, a spring resting against the stubs and the inside of the push-button body respectively, an insulating cylindrical casing conventional for this application, which is both shock and temperature resistant, and of which one end receives the push-button assembly and the percussion mass, and the other end of which receives a conventional impact assembly comprising a striker, a piezoelectric crystal, an anvil, a connection and a lock washer. Briefly, when the push-button is depressed, the spring is compressed, whereupon the stubs move on the inclines until at the end of their travel the percussion mass is abruptly released so that, under the influence of the spring, it impacts the impact assembly and thus generates the spark. Thereupon the mass is returned to its initial position by a suitable return spring. Piezoelectric generators, or igniters, of this kind are known and commercially available. Some examples of particular designs are illustrated in U.S. Pat. Nos. 4,422,124, 3,751,690, and 4,658,508, each of these references is incorporated herein by reference.

In some embodiments, a portable defibrillator is provided which may be constructed from the basic components of a piezoelectric acupuncture device with modifications in the skin contacting electrode so that the skin contact electrode has a planar skin contact surface.

The mechanics and physics behind piezoelectric generators is well known in the art. What has not been known previously is that the electric shock generated by such a device may be used to restore normal contraction rhythm to a heart in cardiac arrest, such as ventricular fibrillation or asystole.

Accordingly, the present invention provides a piezoelectric defibrillator is comprising a housing having an electrically conductive portion; a piezoelectric generator seated within the housing and electrically insulated from the conductive portion of the housing, the piezoelectric generator having first and second electrical conductors, the first conductor being electrically connected to the electrically conductive portion of the housing; an electrically conductive skin contact electrode having a skin contact portion, the electrode being electrically insulated from the conducting portion of the housing, and being in electrical connection with the second conductor; an actuator for actuating the piezoelectric generator to generate an electrical current in response to stimuli from a user. The piezoelectric generator may be a percussion type piezoelectric generator.

The present invention also provides methods of restoring normal rhythm of a heart in cardiac arrest in a patient comprising administering an electric shock from a piezoelectric device to the chest area proximal the heart of the patient.

The present invention also provides the use of a piezoelectric device capable of producing an electrical shock for restoring normal pumping action to a heart in cardiac arrest.

There is also provided a piezoelectric heart pacer comprising a housing having an electrically conducting portion, a piezoelectric generator within the housing and electrically insulated from the housing, the piezoelectric generator having first and second electrical conductors, the first conductor being electrically connected to the electrically conducting portion of the housing, an electrically conductive skin contact electrode having a skin contact portion, the electrode being electrically insulated from the conducting portion of the housing, and being in electrical connection with the second conductor, and an actuator for actuating the piezoelectric generator to generate an electrical current in response to stimuli from a user.

There is further provided methods of pacing a heart comprising the steps of administering an electric shock from a piezoelectric device to the chest area of a mammal proximal the heart and repeating the application of an electric shock as necessary to establish a desired beat rhythm of the heart, and the use of a piezoelectric device capable of producing an electrical shock for pacing a heart.

### BRIEF DESCRIPTION OF THE DRAWINGS

Some embodiments of the invention will now be described with reference to the accompanying drawings, in which.

FIG. 1 is a cross-sectional view of a piezoelectric defibrillator; and

FIG. 2 is a cross-sectional view of a piezoelectric defibrillator showing an alternate embodiment of the skin contact portion.

### DETAILED DESCRIPTION

As indicated above, I made a surprising discovery that the application of an electrical shock from a piezoelectric device, such as a piezoelectric acupuncture device, to the upper portion of the left anterior chest of a patient whose heart is in cardiac arrest (ventricular fibrillation or asystole) has the effect of terminating the chaotic activity of the heart and restore the normal pumping action of the heart. This discovery was made as a result of my having revived patients who had a sudden cardiac arrest in my medical office, by administering electrical shock from a hand-held piezoelectric acupuncture device to the upper portion of the left anterior chest. The device was held in one hand and its treatment head was brought into contact with the patient's bare chest, while my other hand was brought into contact with the patient's bare back to complete the circuit and direct the electrical current through the chest cavity.

Reference will now be made in detail to the present preferred embodiments of the invention, examples of which are illustrated in the accompanying drawings wherein like numerals indicate the same elements throughout the views.

Portable piezoelectric defibrillator 10 comprises an electrically conducting, tubular outer casing or housing 12 within which is located or seated a percussion type piezoelectric generator 14. Piezoelectric generator 14 is electrically insulated from outer casing 12 by either electrically insulator layer 16 or airspace 18, or a combination of the two as illustrated. Piezoelectric generator 14 may be of a design known in the art, such as those described in U.S. Pat. Nos. 4,139,792, 4,422,124 and 4,608,508, with or without modifications to enable the device to generate suitable electrical currents. Each of the aforementioned references is incorporated herein by reference. However, it will now be apparent to a person of ordinary skill in the art that other kinds of piezoelectric generators may be suitable for use in some embodiments of the present invention. As is typical in such piezoelectric generators, two electrical conductors or electrical wires, such as first and second wires 20 and 22, extend from piezoelectric generator 14 and carry the current generated by the device upon actuation. Electrical wire 22 is electrically connected to a ski contact electrode 24 which terminates in a skin contact portion 26. The electrode 24 and skin contact portion 26 are electrically insulated from casing 12 by insulator layer 28, which may be airspace. At the opposite end of defibrillator 10 that is remote from the skin contact portion, actuator or plunger button 30 is provided within the casing and extends out longitudinally beyond the casing. Button 30 mechanically communicates with the mechanical mechanism of the piezoelectric generator. Lead wire 20 is in electrical communication with casing 12, which functions as an electrical ground.

In operation of the piezoelectric defibrillator, an operator holds the defibrillator 10 in one hand with a thumb over the actuator button 30. The skin contact electrode 26 of the defibrillator is brought into contact with the patient's bare skin in the chest area near the patient's heart. The user's other hand is placed on bare skin on the back of the patient underneath the heart. The operator then depresses the actuator button to actate the piezoelectric generator to release a burst of electricity. It is apparent that the operator acts as an electrical ground and completes an electrical circuit since the opertor's hand is in electrical communication with casing 12, which is itself electrically connected to lead wire 20 of generator. Therefore, the operator may direct the path of the electrical current through the patient's chest cavity by the placement of the free hand that is not holding the device on the bare skin of the back of the patient. Thus the electrical current generated by the piezoelectric generator travels through lead wire 22, electrode 24, skin contact portion 26, through the patient's chest cavity and heart, through the operator's arms and hands.

While the piezoelectric defibrillator of the present invention has described to be used on a human patient, it is to be noted that the device may be used on any mammal.

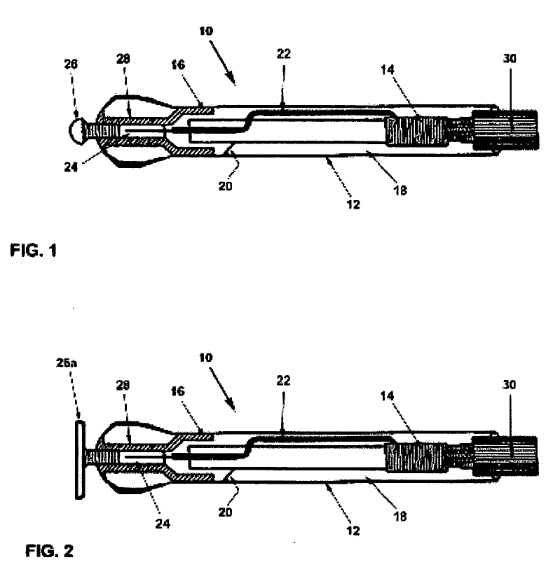

Referring to FIG. 2, there is illustrated an alternate embodiment of the present invention in which the portable defibrillator 10 is similar to that in FIG. 1 except that the a wider skin contacting portion 26a is provided which has a generally planar skin contact surface to distribute the electrical charge more widely at the contact point. For example, skin contact portion 26a may be of similar general construction as electrodes of conventional defibrillator.

It is known that in piezoelectric devices which generate an electrical shock, a hammer or striker is suddenly forced, usually by some biasing or spring mechanism, against the piezoelectric generator. The compression of the piezoelectric generator generates an electrical charge. The parameters of the charge may be controlled by the selection of the piezoelectric generator and by the magnitude of the impact of the hammer against the piezoelectric generator. The piezoelectric generator and the adjustment of the force of the impact hammer should be selected so that the electrical charge generated is sufficient for the purposes of restoring normal contraction rhythms, or defibrillating, a heart. The parameters of suitable electric charges are known in the art.

In administering electric shock to the chest area, the piezoelectric defibrillator is held in one hand of the user who is administering the electrical shock to the patient whose heart is experiencing cardiac arrest. The electrode of the piezoelectric defibrillator is brought into contact with exposed skin of the upper portion of the left anterior chest of the patient near the patient's heart. The user's other bare hand should be brought into contact with exposed skin of the patient's back. Thus the other hand functions as a ground to direct electrical cr through the patient's heart. The user then discharges the piezoelectric defibrillator to deliver an electrical shock to the patient's chest as often as is necessary to defibrillate the heart.

The foregoing description of the preferred embodiments of the invention has been presented for purposes of illustration and description. It is not intended to be exhaustive or to limit the invention to the precise form disclosed. Modifications or variations are possible and contemplated in light of the above teachings by those skilled in the ad, and the embodiments discussed were chosen and described in order to best illustrate the principles of the invention and its practical application. It is intended that the scope of the invention be defined by the claims appended hereto.

1. A piezoelectric defibrillator comprising:
a housing having an electrically conductive portion;
a piezoelectric generator seated within the housing and electrically insulated from the conductive portion of the housing, the piezoelectric generator having first and second electrical conductors, the first conductor being electrically connected to the electrically conductive portion of the housing;
an electrically conductive skin contact electrode having a skin contact portion, the electrode being electrically insulated from the conducting portion of the housing, and being in electrical connection with the second conductor;
an actuator for actuating the piezoelectric generator to generate an electrical current in response to stimuli from a user.

2. An apparatus as in claim 1 wherein the piezoelectric generator is a percussion type piezoelectric generator.

3. An apparatus as in claim 2 wherein the skin contact portion is planar.

4. The use of electric current from a piezoelectric device for restoring normal contraction rhythm to a heart in cardiac arrest in a mammal.

5. The use in claim 4 wherein the piezoelectric device is a piezoelectric acupuncture device.

6. A method of restoring normal contraction rhythm to a heart in cardiac arrest in a mammal, the method comprising the step of administering an electric shock from a piezoelectric device to the chest area proximal the heart of the mammal.

7. The method as in claim 6 wherein the piezoelectric device is held in one hand by the user, and further comprising the step of the user placing his or her other hand on a portion of skin of the mammal to act as a ground for electrical current generated by the piezoelectric device in such a way as to direct the current from the piezoelectric device to flow through the heart.

8. A method as in claim 7 wherein the piezoelectric device comprises a piezoelectric acupuncture device.

9. A method as in claim 7 wherein the piezoelectric device comprises an apparatus as claimed in claim 2.

10. A piezoelectric device for manually pacing a heart in a mammal, the device comprising:
a housing having an electrically conductive portion;
a piezoelectric generator seated within the housing and electrically insulated from the conductive portion of the housing, the piezoelectric generator having first and second electrical conductors, the first conductor being electrically connected to the electrically conducting portion of the housing;
an electrically conductive skin contact electrode having a skin contact portion, the electrode being electrically insulated from the conducting portion of the housing, and being in electrical connection with the second conductor;
an actuator for actuating the piezoelectric generator to generate an electrical current in response to stimuli from a user.

11. An apparatus as in claim 10 wherein the piezoelectric generator is a percussion type piezoelectric generator.

12. The use of electric current from a piezoelectric device for manually pacing a heart in a mammal.

13. The use in claim 12 wherein the piezoelectric device is a piezoelectric acupuncture device.

14. A method of pacing a heart in a mammal comprising the steps of administering an electric shock from a piezoelectric device to the chest area of the mammal proximal the heart, and repeating the application of an electric shock as necessary to maintain a desired beat rhythm of the heart.

15. The method as in claim 14 wherein the piezoelectric device is held in one hand by the user, and further comprising the step of the user placing his or her other hand on a portion of skin of the mammal to act as a ground for electrical current generated by the piezoelectric device in such a way as to direct the current from the piezoelectric device to flow through the heart.

16. A method as in claim 15 wherein the piezoelectric device comprises a piezoelectric acupuncture device.
